(19) United States
(12) Patent Application Publication
(10) Pub. No.: US 2016/0136057 A1
(43) Pub. Date: May 19, 2016

(54) SYSTEM AND APPARATUS FOR CONTROLLING FOOD INTAKE

(71) Applicant: UMM AL-QURA UNIVERSITY, Makkah (SA)

(72) Inventor: Muhammad ARIF, Makkah (SA)

(73) Assignee: UMM AL-QURA UNIVERSITY, Makkah (SA)

(21) Appl. No.: 14/405,009

(22) PCT Filed: Nov. 19, 2014
(86) PCT No.: PCT/IB2014/002509

§ 371 Date: Dec. 2, 2014

(51) Int. Cl. A61J 15/00; A61B 5/00

(52) U.S. Cl. CPC A61J 15/0088; A61J 15/0049; A61B 5/4866; A61J 2200/76

(57) ABSTRACT

A controlled food intake system includes an apparatus having an inlet tube configured to connect to a lower esophageal stoma, a flow controller, and at least one container configured to detachably attach to the inlet tube and to administer nutrition liquids to a patient at a flow rate based on a position of the flow controller. The apparatus also includes an outlet tube configured to connect to an upper esophageal stoma and a waste container that detachably attaches to the outlet tube. Processing circuitry of the apparatus is configured to control the flow controller to control the flow rate of the nutrition liquids being administered to the patient based on prescribed nutrition intake order. The controlled food intake system also includes at least one server configured to generate reports based on the nutrition liquids administered to the patient and substance consumed by the patient.

### BACKGROUND

The “background” description provided herein is for the purpose of generally presenting the context of the disclosure. Work of the presently named inventor, to the extent it is described in this background section, as well as aspects of the description which may not otherwise qualify as prior art at the time of filing, are neither expressly nor impliedly admitted as prior art against the present disclosure.

Impacts of diseases affected by a person's diet, such as hypertension, diabetes, obesity, and the like, have been increasing over the last several decades. Modifying dietary habits to alleviate the effects of such diseases can be difficult for patients, and it may take a long period of time to see the effects of the dietary changes.

### SUMMARY

In an exemplary embodiment, a controlled food intake system includes an apparatus having an inlet tube configured to connect to a lower esophageal stoma, a flow controller, and at least one container configured to detachably attach to the inlet tube and to administer nutrition liquids to a patient at a flow rate based on a position of the flow controller. The apparatus also includes an outlet tube configured to connect to an upper esophageal stoma and a waste container that detachably attaches to the outlet tube. Processing circuitry of the apparatus is configured to control the flow controller to control the flow rate of the nutrition liquids being administered to the patient based on prescribed nutrition intake order. The controlled food intake system also includes at least one server configured to generate reports based on the nutrition liquids administered to the patient and substance consumed by the patient.

The foregoing general description of exemplary implementations and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates an exemplary controlled food intake system, according to certain embodiments;

FIG. 2 illustrates an exemplary controlled food intake apparatus, according to certain embodiments;

FIG. 3 is an exemplary illustration of one or more inflators for a controlled food intake apparatus, according to certain embodiments;

FIG. 4 illustrates an exemplary controlled food intake process, according to certain embodiments;

FIG. 5 illustrates an exemplary waste collection process, according to certain embodiments;

FIG. 6 is an exemplary illustration of a display for a controlled food intake apparatus controller, according to certain embodiments;

FIG. 7A is an exemplary illustration of a patient order screen, according to certain embodiments;

FIG. 7B is an exemplary illustration of a patient meal input screen, according to certain embodiments;

FIG. 7C is an exemplary illustration of a controlled food intake report, according to certain embodiments; and

FIG. 8 illustrates a non-limiting example of a controlled food intake apparatus controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise. The drawings are generally drawn to scale unless specified otherwise or illustrating schematic structures or flowcharts.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to controlling food intake of a patient with one or more medical conditions that preclude a type or quantity of food intake, such as obesity, diabetes, digestive system disorders, and the like. A controlled food intake apparatus can be attached to the patient's esophagus via two surgical incisions which allows the patient to consume foods that may not be in the patient's restricted diet while still maintaining nutrition standards based on the medical condition. In certain embodiments, the controlled food intake apparatus is used to wean the patient off foods that do not conform to the nutrition standards. Details regarding the controlled food intake apparatus are discussed further herein.

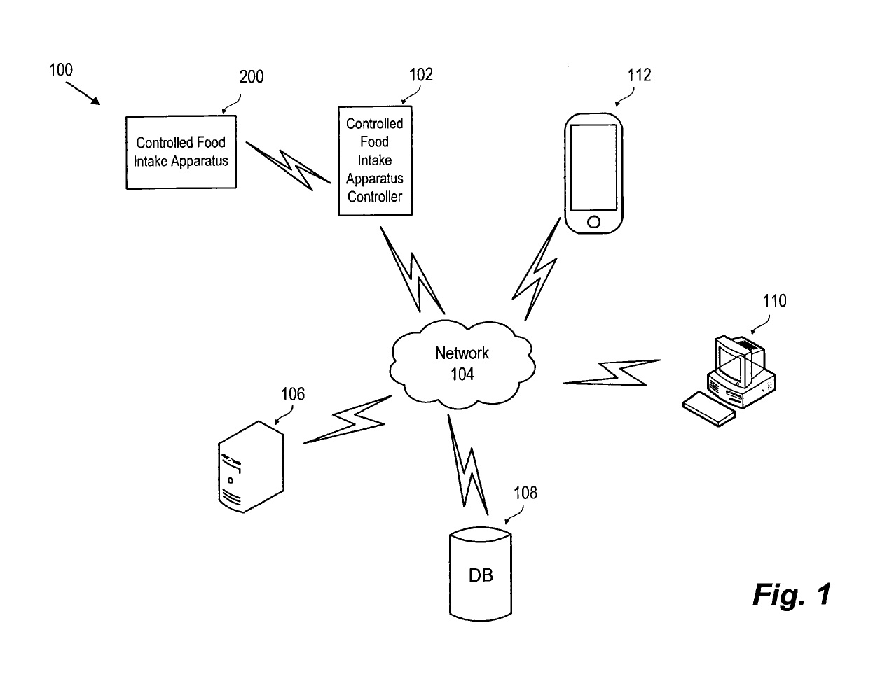

FIG. 1 illustrates an exemplary controlled food intake system 100, according to certain embodiments. A controlled food intake apparatus controller 102 is connected to a server 106, database 108, computer 110, and mobile device 112 via a network 104. Throughout the disclosure, the controlled food intake apparatus controller 102 is interchangeably referred to as the controller 102. The computer 110 acts as a client device that is connected to the server 106, the database 108, the mobile device 112, and the controller 102 via the network 104. The server 106 represents one or more servers connected to the computer 110, the database 108, the mobile device 112, and the controller 102 via the network 104. The database 108 represents one or more databases connected to the computer 110, the server 106, the mobile device 112, and the controller 102 via the network 104.

The mobile device 112 represents one or more mobile devices connected to the computer 110, the server 106, the database 108, and the controller 102 via the network 104. The network 104 represents one or more networks, such as the Internet, connecting the computer 110, the server 106, the database 108, the mobile device 112, and the controller 102. In some embodiments, more than one controller 102 is included in the controlled food intake system 100. As such, the terms referring to the one or more than one controller 102 can be used interchangeably.

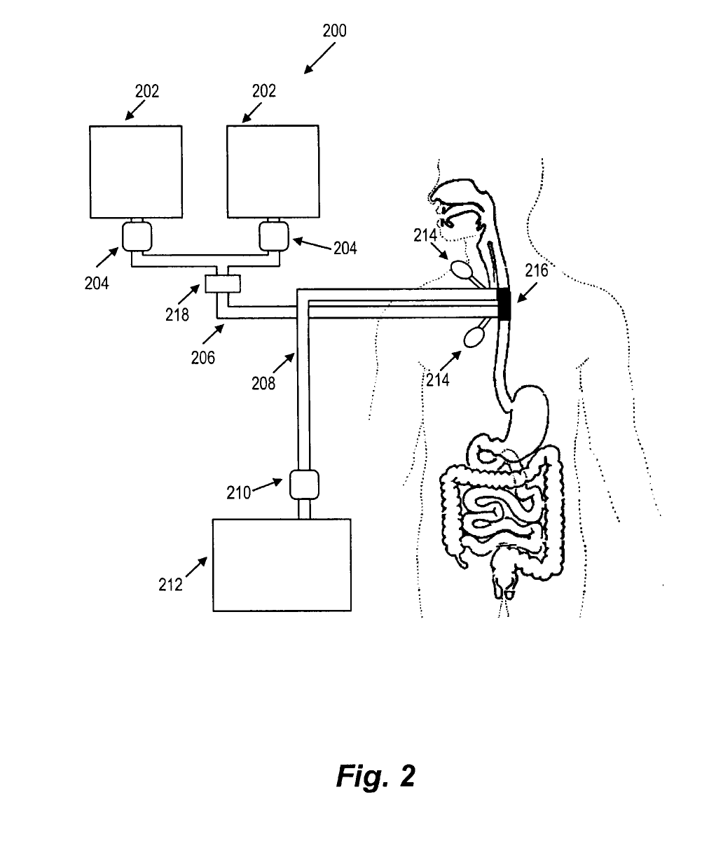

In certain embodiments, the controller 102 performs wireless or wired communications with at least one controlled food intake apparatus 200 (illustrated in FIG. 2). The controller 102 and the controlled food intake apparatus 200 can communicate via wireless networks such as WI-FI, BLUETOOTH, cellular networks including EDGE, 3G and 4G wireless cellular systems, or any other wireless form of communication that is known.

As would be understood by one of ordinary skill in the art, based on the teachings herein, the mobile device 112 or any other external device could also be used in the same manner as the computer 110 to receive inputs from medical providers and/or patients. For example, a medical provider, such as a physician, inputs nutrition intake orders for the patient at an interface at the computer 110 or via an application on the mobile device 112. The medical provider can input types of nutrition liquids to be administered to the patient, daily caloric intake, number of feedings per day, flow rate, and the like. In addition, the patient can input types and quantities of foods consumed by mouth and times of meals at the interface at the computer or via the mobile device 112. The information input at the computer 110 or mobile device 112 is received by the server 106 and is processed by processing circuitry of the server 106 and/or controller 102 to determine parameters for administering the nutrition liquids through the controlled food intake apparatus 200. The server 106 also generates reports based on the operation of the controlled food intake apparatus 200 and the food consumed by the patient. Details regarding the processes performed by the controlled food intake system 100 are discussed further herein.

FIG. 2 illustrates an exemplary controlled food intake apparatus 200, according to certain embodiments. Nutrition liquids and fluids, such as water, that are to be provided to the patient are added to one or more nutrition containers 202. In certain embodiments, the nutrition liquids can include nutrition shakes, liquid supplements, and the like. The nutrition containers 202 can include level detectors that detect a level of liquid remaining in the nutrition containers 202. If the level of liquid in a nutrition container 202 reaches at least one predetermined level, a control signal is sent to the controller 102 to output an alarm that warns the medical provider and/or patient that an amount of liquid is remaining in a nutrition container 202. The alarm that is emitted from the controller 102 can be audible, visual, and/or tactile or can be communicated to the medical provider at a computer 110 or mobile device 112 via the network 104. In some implementations, one predetermined level of liquid is set to correspond to when the nutrition container 202 is approximately empty. Another predetermined level of liquid can be set to correspond to a volume of liquid remaining in the nutrition container 202 that is less than an amount of liquid that is required to be administered to the patient during a feeding.

At least one inlet flow controller 204 controls the flow rate of fluid through an inlet tube 206. The inlet flow controller 204 includes a proportional flow valve, a manual valve operator, and circuitry to process control signals from the controller 102 regarding a flow rate of nutrition liquids or other fluids to administer to the patient when the controlled food intake apparatus 200 is in an automatic (auto) or override mode of operation. Based on the control signal, the inlet flow controller 204 modifies how far the proportional flow control valve opens to administer the nutrition liquids to the patient at the flow rate determined by the controller 102. For example, based on the flow rate input by the medical provider at the interface of the computer 110 or mobile device 112, the processing circuitry of the controller 102 determines a percentage from fully open for the proportional flow valve in order to deliver the nutrition liquid to the patient at the prescribed flow rate. The controller 102 then sends a control signal to the inlet flow controller 204 to open the proportional flow valve to the percentage from fully open determined by the processing circuitry.

In certain embodiments, the controlled food intake apparatus 200 has a manual mode of operation where components of the controlled food intake apparatus 200 are controlled by a user, such as the medical provider. The controller 102 remains passive in the manual mode of operation, which means that the controller does not issue control signals to control the controlled food intake apparatus 200. The medical provider manually adjusts the manual valve operator of the one or more inlet flow controllers 102 to achieve the desired flow rate through the inlet tube 206. The controlled food intake apparatus 200 also has an override mode of operation where the medical provider or person administering the nutrition liquids can override a prescribed flow rate and/or other directions determined by the prescribing medical provider and set the flow rate at the controller 102. In each mode of operation of the controlled food intake apparatus 200, the controller 102 can display a flow rate that corresponds to the percentage that the proportional flow valve is from fully open. (Details regarding the display for the controller 102 are discussed further herein.) For example, a proportional flow valve that is 35% open can correspond to a flow rate of 300 milliliters/hour (mL/hr). The flow rate determined by the percentage that the proportional flow valve is from fully open can be referred to as an ordered flow rate. If there is more than one nutrition container 202 and more than one inlet flow controller 204, the ordered flow rate can be determined to be an average of the ordered flow rates of the inlet flow controllers 204.

An inlet flowmeter 218 is located within the inlet tube 206 and is downstream of the at least one inlet flow controller 204, according to certain embodiments. The inlet flowmeter 218 can be any type of flowmeter than can measure the flow rate of the nutrition liquids passing through the inlet tube 206, such as a mechanical flowmeter, an optical flowmeter, a pressure flowmeter, and the like. In addition, the inlet flowmeter 218 can measure volumetric flow rate or mass flow rate. In some implementations, the flow rate measured by the inlet flowmeter 218 corresponds to an actual or measured flow rate through the inlet tube 206 and can be displayed on the controller 102. If a difference between the measured flow rate measured by the inlet flowmeter 218 and the ordered flow rate at the inlet flow controller 204 is greater than a predetermined threshold, then the controller 102 can issue an alarm that is audible, visual, and/or tactile to alert the medical providers. A difference between the measured and ordered flow rates that is greater than the predetermined threshold may indicate that there is a blockage in the inlet tube 206.

The inlet tube 206 is inserted into the esophagus 216 of the patient through a lower esophageal stoma that is made during a surgical procedure. The lower esophageal stoma is a surgically created orifice that creates an entrance for the inlet tube 206 to enter the esophagus 216 of the patient. The nutrition liquids flow from the nutrition container 202, through the inlet tube 206 and into the esophagus 216 via the lower esophageal stoma. Once in the esophagus, the nutrition liquids can flow directly into the stomach to be digested. One or more inflators 214 are attached to the end of the inlet tube 206 that is inserted into the lower esophageal stoma to ensure that the inlet tube 206 is tightly sealed to the wall of the esophagus 216. Details regarding the one or more inflators 214 are discussed further herein. In certain embodiments, the inlet tube 206 is made of flexible material, such as rubber, silicon, polyurethane, and the like and has a diameter within the range of one to ten millimeters (mm) that enables the inlet tube 206 to be inserted into the lower esophageal stoma.

In certain embodiments, the nutrition containers 202 are positioned above the location of the lower esophageal orifice so that the nutrition liquids flow through the inlet tube 206 due to gravitational force. If the nutrition containers 202 are positioned at a level in relation to the lower esophageal stoma that limits gravitational flow of the nutrition liquids, a pump can be added between each nutrition container 202 and the corresponding inlet flow controller 204 to ensure that the nutrition liquids are able to reach the esophagus 216. The pump can be a centrifugal pump, positive displacement pump, or the like.

An outlet tube 208 is inserted into the esophagus 216 of the patient through an upper esophageal stoma that is made during a surgical procedure. The upper esophageal stoma is a surgically created orifice that creates an entrance for the outlet tube 208 to enter the esophagus 216 of the patient and is positioned above the lower esophageal stoma, according to certain embodiments. In some implementations, the patient consumes food and liquids through the mouth and swallows the predigested food, which travels through the esophagus 216 and into the outlet tube 208 via the upper esophageal stoma. The predigested food then flows through the outlet tube 208 and into a waste container 212. Therefore, a patient who is suffering from a medical condition that restricts food intake can the types and quantities of food that the patient desires because the pre-digested food exits the body through the upper esophageal stoma.

The waste container 212 is detachably attached to a distal end of the outlet tube 208 downstream of an outlet flowmeter 210 so that the waste container 212 can be detached from the outlet tube 208 to discard the contents after the waste container 212 becomes full of predigested food. In certain embodiments, the waste container 212 includes a weight sensor to determine how much food has been consumed by the patient. The waste container 212 can also include a food filter (not shown) that separates the predigested food that may have a higher viscosity than liquids that are consumed, such as water, so that it can be estimated how much food and liquid have been consumed by the patient. The waste container 212 can also include at least one level detector that indicates a fullness of the waste container 212. When the level of the waste container 212 reaches a predetermined threshold that indicates that the waste container is almost full and should be emptied, an alarm can be output at the controller 102 and can be audible, visual, and/or tactile.

The outlet flowmeter 210 is located within the outlet tube 208, according to certain embodiments. The outlet flowmeter 210 can be any type of flowmeter than can measure the flow rate of the nutrition liquids passing through the outlet tube 208, such as a mechanical flowmeter, an optical flowmeter, a pressure flowmeter, and the like. In addition, the outlet flowmeter 218 can measure volumetric flow rate or mass flow rate. In certain embodiments, the outlet flowmeter 210 can be used to determine a mass or volume of food that has been consumed through the mouth of the patient.

One or more inflators 214 are attached to the end of the outlet tube 208 that is inserted into the upper esophageal stoma that ensures that the outlet tube 208 is tightly sealed to the wall of the esophagus 216. Details regarding the one or more inflators 214 are discussed further herein. In certain embodiments, the outlet tube 208 is made of flexible material, such as rubber, silicon, polyurethane, and the like and has a diameter within the range of one to ten millimeters (mm) that enables the outlet tube 208 to be inserted into the upper esophageal stoma.

In certain embodiments, the waste container 212 is positioned below the location of the upper esophageal orifice so that the predigested food flows through the outlet tube 208 due to gravitational force. If the waste container 212 is positioned at a level in relation to the upper esophageal stoma that limits gravitational flow of the predigested food, a pump can be attached in the flow path of the outlet tube 208 to ensure that the predigested food is able to reach the waste container 212. The pump can be a centrifugal pump, positive displacement pump, or the like.

In some implementations where the patient is unable to consume food and liquids by mouth, the outlet tube 208, outlet flowmeter 210, and waste container 212 may not be included in the controlled food intake apparatus 200. In addition, the upper esophageal stoma may not have to be surgically created. The patient can still receive nutrition liquids via the nutrition containers 202 and the inlet tube 206 that enters the esophagus 216 of the patient through an esophageal stoma.

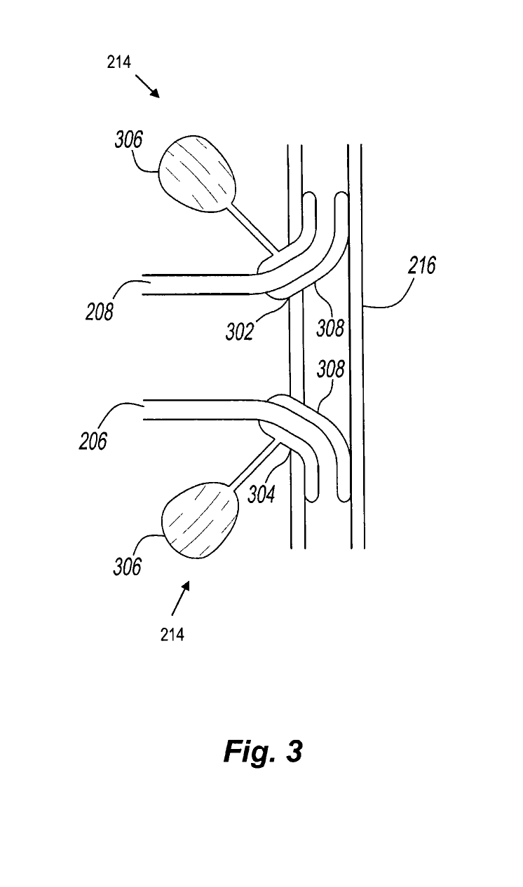

FIG. 3 is an exemplary illustration of the one or more inflators 214 for a controlled food intake apparatus 200, according to certain embodiments. An inflator 214 is attached to the proximal end of the inlet tube 206, and an inflator 214 is also attached to the proximal end of the outlet tube 208 to ensure that the inlet tube 206 and the outlet tube 208 are tightly sealed to the wall of the esophagus 216 when fully inserted into the lower esophageal stoma 304 and the upper esophageal stoma 302. The one or more inflators 214 include a hand pump 306 that allows a medical provider to push air into a tube housing 308 to create a seal with the lower esophageal stoma 304 or the upper esophageal stoma 302. In certain embodiments, the one or more inflators 214 also include a deflator port (not shown) that enables air to be released from the inflator 214 prior to insertion or removal of the inlet tube 206 or outlet tube 208 from the lower esophageal stoma 304 or the upper esophageal stoma 302. The deflator port can be a hole installed in the one or more inflators 214 that provides a path for air to escape when deflating the one or more inflators 214 and is covered by a plug or screw when the one or more inflators 214 are filled with air.

In addition, a pressure sensor can be installed within the tube housing 308 that detects when air pressure within the inflator 214 is less than a predetermined threshold that may indicate that air has escaped from the inflator 214 and the seal with the esophageal stoma may have been lost. If the pressure detected by the pressure sensor in the tube housing 308 is less than the predetermined threshold, an inflator low pressure alarm may be emitted at the controller 102 that is audible, visible, and/or tactile.

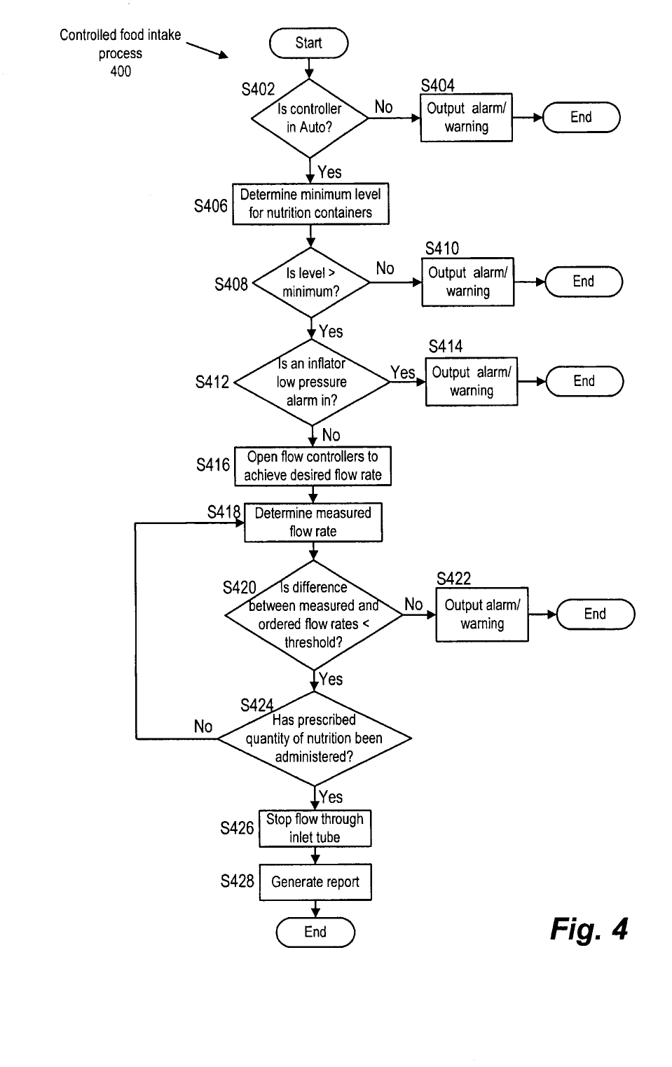

FIG. 4 illustrates an exemplary controlled food intake process 400, according to certain embodiments. The controlled food intake process 400 can be performed when the controller 102 is set to the auto mode of operation, which means that the processing circuitry of the controller 102 manages the administering of nutrition liquids to the patient via the controlled food intake apparatus 200. In addition, the controlled food intake process 400 commences when the medical provider or patient activates a button or switch at the controller 102 to initiate the process. At step S402, a determination is made whether the controller 102 is set to auto. If the controller 102 is in auto, resulting in a “yes” at step S402, then step S406 is performed. Details regarding the auto mode of operation are discussed further herein.

Otherwise, if the controller 102 is set to manual or override, resulting in a “no” at step S402, step 5404 is performed. At step S404, the controller 102 outputs an alarm and/or warning to alert the medical provider and/or patient that the controller 102 is not in auto and that the controlled food intake process 400 will be terminated. The alarm can be audible, visual, and/or tactile, and a warning can be displayed on a message screen of the controller 102. After step S404 is performed, the controlled food intake process 400 is terminated. By alerting the medical provider and/or patient that the controller 102 is not in auto, the medical provider and/or patient may be prompted to place the controller 102 in auto in order to administer nutrition liquids to the patient.

At step S406, the processing circuitry of the controller 102 determines a minimum initial level for the nutrition containers 202 based on a quantity of nutrition liquids to be administered to the patient. For example, in some implementations, the prescribing medical provider can determine the quantity of nutrition liquids per meal or per day. Based on the dimensions of the nutrition containers 202, the processing circuitry can determine a minimum initial level of nutrition liquids that can be present in the nutrition containers 202 in order to administer the prescribed quantity of nutrition liquids to the patient.

At step S408, it is determined if a current level of nutrition liquids in the nutrition containers 202 detected by the level detectors is greater than the minimum initial level determined at step S406. If the current level of nutrition liquids in the nutrition containers 202 is greater than the minimum initial level, resulting in a “yes” at step S408, then step S412 is performed. Otherwise, if the current level of nutrition liquids in the nutrition containers 202 is less than or equal to the minimum initial level, resulting in a “no” at step S408, then step S410 is performed. If the current level of nutrition liquids in the nutrition containers 202 is less than the minimum initial level, then the nutrition containers 202 may become empty before the controlled food intake apparatus 200 has administered the prescribed quantity of nutrition liquids to the patient.

At step S410, the controller 102 outputs an alarm and/or warning to alert the medical provider and/or patient that the quantity of nutrition liquids in the nutrition containers 202 is less than the prescribed quantity of nutrition liquids to be administered to the patient. The alarm can be audible, visual, and/or tactile, and a warning can be displayed on a message screen of the controller 102. After step S410 is performed, the controlled food intake process 400 is terminated. By alerting the medical provider and/or patient that the quantity of nutrition liquids in the nutrition containers 202 is less than the prescribed quantity of nutrition liquids to be administered to the patient, the medical provider and/or patient may be prompted to add additional nutrition liquids to the nutrition containers 202.

At step S412, it is determined whether an inflator low pressure alarm is activated. In certain embodiments, a pressure sensor can be installed within the tube housing 308 of the inflator 214 that detects when air pressure within the inflator 214 is less than a predetermined threshold that may indicate that air has escaped from the inflator 214 and the seal with the esophageal stoma may have been lost. If the pressure detected by the pressure sensor in the tube housing 308 is less than the predetermined threshold, an inflator low pressure alarm may be emitted at the controller 102 that is audible, visible, and/or tactile. If the inflator low pressure alarm is not activated, resulting in a “no” at step S412, then step S416 is performed. Otherwise, if the inflator low pressure alarm is activated, resulting in a “yes” at step S412, then step S414 is performed.

At step S414, the controller 102 outputs an alarm and/or warning to alert the medical provider and/or patient that the inflator low pressure alarm is activated. The alarm can be audible, visual, and/or tactile, and a warning can be displayed on a message screen of the controller 102. After step S414 is performed, the controlled food intake process 400 is terminated. By alerting the medical provider and/or patient that the inflator low pressure alarm is activated, the medical provider and/or patient may be prompted to add more air to the inflator 214 for the inlet tube 206 or the outlet tube 208 to clear the inflator lower pressure alarm and ensure that a seal exists between the inflator 214 and the upper esophageal stoma 302 and/or the lower esophageal stoma 304.

At step S416, the controller 102 issues control signals to open the proportional flow valve of the at least one inlet flow controller 204 to a percentage from fully open that corresponds to the ordered flow rate that is determined by the prescribing medical provider, as will be discussed further herein. For example, if the ordered flow rate is 300 mL/hr, the proportional flow valve may be opened to 35% to achieve the prescribed flow rate. If there is more than one nutrition container 202 and more than one inlet flow controller 204, the ordered flow rate can be determined to be an average of the ordered flow rates of the inlet flow controllers 204.

At step S418, a measured flow rate detected at the inlet flowmeter 218 is obtained by the controller 102. In certain embodiments, measured flow rates and an associated times that are obtained during the administration of nutrition liquids to the patient are stored in the database 108 and can be used to determine a total amount of nutrition liquids that are administered to the patient. In some implementations, the flow rate measured by the inlet flowmeter 218 corresponds to an actual flow rate through the inlet tube 206.

At step S420, it is determined whether a difference between the measured and ordered flow rates through the inlet tube 206 is less than a predetermined threshold. If the difference between the measured and ordered flow rates is less than the predetermined threshold, resulting in a “yes” at step S420, then step S424 is performed. Otherwise, if the difference between the measured flow rate detected by the inlet flowmeter 218 and the ordered flow rate at the inlet flow controller 204 is greater or equal to the predetermined threshold, resulting in a “no” at step S420, then step S422 is performed.

At step S422, the controller 102 outputs an alarm and/or warning to alert the medical provider and/or patient that the difference between the measured and ordered flow rates within the inlet tube 206 is greater than or equal to the predetermined threshold. The alarm can be audible, visual, and/or tactile, and a warning can be displayed on a message screen of the controller 102. After step S422 is performed, the controlled food intake process 400 is terminated. A difference between the measured and ordered flow rates that is greater than the predetermined threshold may indicate that there is a blockage in the inlet tube 206, and the alarm and/or warning that are output at the controller 102 can notify the medical provider and/or patient to check for blockages in the inlet tube 206.

At step S424, the processing circuitry of the controller 102 determines if the prescribed quantity of nutrition liquids have been administered to the patient via the controlled food intake apparatus 200. In some implementations, the prescribing medical provider prescribes a quantity of nutrition liquids to be administered to the patient. Based on the measured flow rate of the inlet flowmeter 218, the quantity of nutrition liquids that have been administered to the patient can be determined. If the quantity of nutrition liquids that have been administered to the patient is greater than or equal to the prescribed quantity of nutrition liquids, resulting in a “yes” at step S424, then step S426 is performed. Otherwise, if the quantity of nutrition liquids that have been administered to the patient is less than the prescribed quantity of nutrition liquids, resulting in a “no” at step S424, then the controlled food intake process 400 returns to step S418, and the nutrition liquids continue to be administered to the patient.

At step S426, the proportional control valve of the at least one inlet flow controller is closed in order to stop flow through the inlet tube 206, and nutrition liquids are no longer administered to the patient via the controlled food intake apparatus 200. At step S428 a report is generated by the server 106 regarding the quantity and type of nutrition liquids administered to the patient during a time period. The quantity of nutrition liquids administered to the patient can be based on initial and final levels of liquid in the nutrition containers 202 as well as measured flow rate through the inlet flowmeter 218. The report can also include the nutrition information of the nutrition liquids administered to the patient, such as total calories received, quantity of protein, carbohydrates, fat, and the like. In some implementations, medical providers can receive and view the reports at a computer 110 or mobile device 112.

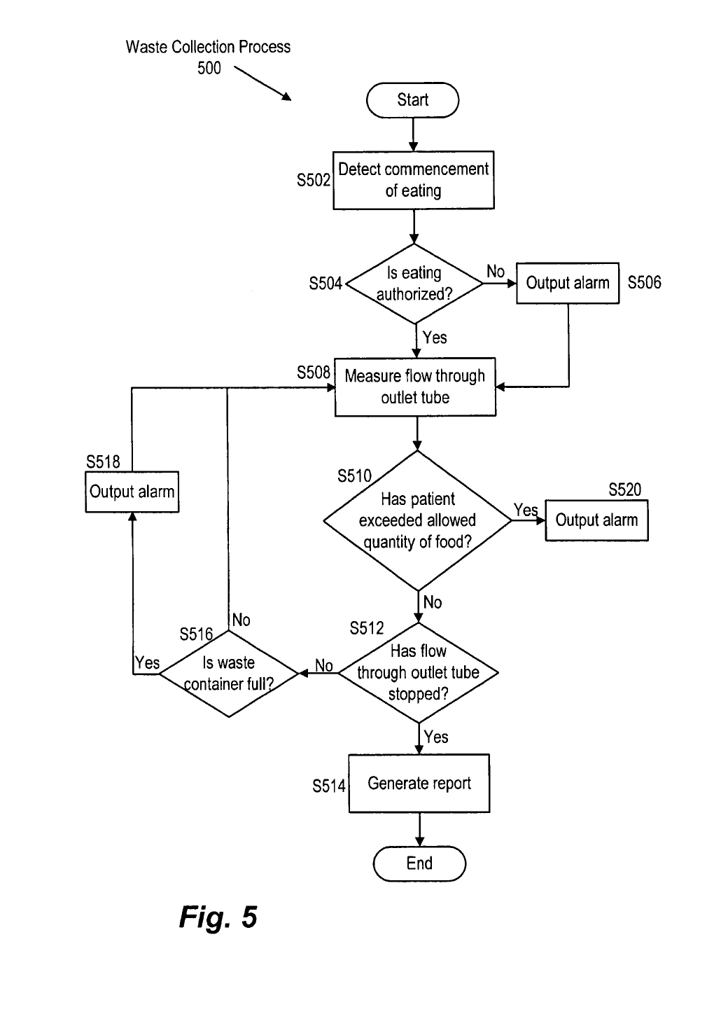

FIG. 5 illustrates an exemplary waste collection process 500, according to certain embodiments. At step S502, the processing circuitry of the controller 102 detects that the patient has commenced eating food through the patient's mouth. In some implementations, the patient or medical provider can push a button on the controller 102 or a remote control that conducts wired or wireless communications with the controller 102 to indicate that the patient is beginning to eat. The food intake apparatus controller 102 can also detect that eating has commenced by detecting flow through the outlet flowmeter 210.

At step S504, it is determined whether eating is authorized. In certain embodiments, the prescribing medical provider may indicate that the patient is authorized at one or more times during a day. In some implementations, the prescribing medical provider may indicate a number of meals that may be spaced at any interval during the day. If the patient is authorized to consume food based on the time of day or number of meals already consumed that day, resulting in a “yes” at step S504, then step S508 is performed. Otherwise, if a current time is not an authorized time for the patient to consume food or if the patient has already consumed the authorized number of meals during the day, resulting in a “no” at step S504, then step S506 is performed. At step S506, the controller 102 outputs an alarm to warn the patient and/or medical provider that the patient is consuming food during an unauthorized time. The alarm may be audible, visual, and/or tactile.

At step S508, the flow through the outlet tube 208 is measured based on the flow rate detected by the outlet flowmeter 210. The measured flow rate may be a volumetric flow rate, mass flow rate, and the like. The controller 102 can acquire the flow rate through the outlet flowmeter 210 at a predetermined sampling rate and store the measured flow rate during the time that the patient is eating in the database 108. In some implementations, the flow rate can be used to determine a mass or volume of food that the patient has consumed during a meal.

At step S510, the processing circuitry of the controller 102 determines if the amount of food consumed by the patient during the meal has exceeded a quantity of food specified by the prescribing medical provider. In certain embodiments, a physician or nutritionist determine allowed quantities of food that the patient should consume by mouth per meal or per day. For example, if the patient is suffering from obesity, the prescribing medical provider may slowly reduce the allowed quantity of food per meal for the patient to consume to enforce a habit of consuming a smaller quantity of food. The amount of food consumed by the patient can be based on the measured flow rate through the outlet flowmeter 210 and/or the mass or volume of predigested food in the waste container 212.

If the quantity of food consumed by the patient during a meal has not exceeded the allowed quantity of food per meal, and if the quantity of food consumed by the patient during a day has not exceeded the allowed quantity of food per day, resulting in a “no” at step S510, then step S512 is performed. Otherwise, if the amount of food consumed by the patient has exceeded the allowed quantity of food per day or per meal, resulting in a “yes” at step S510, then step S520 is performed. At step S520, the controller 102 outputs an alarm to notify the patient and/or medical provider that the amount of food consumed by the patient has exceeded the allowed quantity of food per day or per meal. The alarm may be audible, visual, and/or tactile.

At step S512, the processing circuitry of the controller 102 determines if flow through the outlet tube 208 has stopped, which may indicate that the patient is no longer consuming food through the mouth. It is determined that flow through the outlet tube 208 has stopped if the flow rate detected by the outlet flowmeter 210 decreases to approximately zero. If it is determined that low through the outlet tube 208 has stopped, resulting in a “yes” at step S512, then step S514 is performed. Otherwise, if flow through the outlet tube 208 has not stopped, resulting in a “no” at step S512, then step S516 is performed.

At step S514, a report is generated by the server 106 regarding the quantity of food consumed by the patient during a time period. The report can include time periods during which the patient was consumed. The report can also include a total volume or mass of food consumed by the patient during the time periods based on the flow rate through the outlet flowmeter 210 and/or the mass and/or volume of the contents of the waste container 212. In certain embodiments, the patient can input types and quantities of foods consumed by mouth at an interface at a computer 110 or mobile device 112. The types and quantities of food input by the patient can also be included in the report. In some implementations, medical providers can receive and view the reports at a computer 110 or mobile device 112.

Step S516 is performed if it is determined at step S512 that flow through the outlet tube 208 has not stopped, which may indicate that the patient is still consuming food through the mouth. At step S516, it is determined whether the at least one level detector of the waste container 212 has reached the predetermined threshold indicating that the waste container 212 is full and may need to be emptied. If the at least one level detector of the waste container 212 is less than the predetermined threshold, indicating that the waste container 212 is not full, then a “no” at step S516 results. The waste collection process 500 returns to step S508, and the flow through the outlet tube 208 continues to be measured.

Otherwise, if the at least one level detector of the waste container 212 is greater than or equal to the predetermined threshold, indicating that the waste container 212 is full, then a “yes” at step S516 results, and step S518 is performed. At step S518, the controller 102 outputs an alarm to notify the patient and/or medical provider that the waste container 212 is full and may need to be emptied. The alarm may be audible, visual, and/or tactile. The waste collection process 500 then returns to step S508, and the flow through the outlet tube 208 continues to be measured.

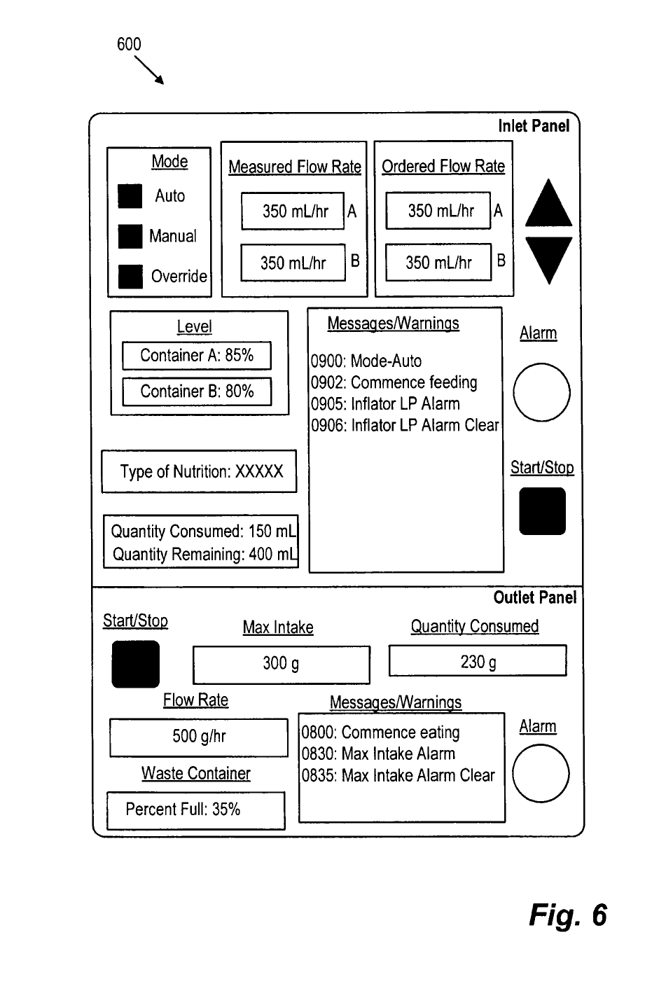

FIG. 6 is an exemplary illustration of a display 600 for a controlled food intake apparatus controller 102, according to certain embodiments. The display can include one or more touchscreens, monitors, and/or pushbuttons that allow a user, such as the medical provider, to make selections and/or inputs at the controller 102. The display 600 includes an inlet panel that displays information and receives inputs regarding administering nutrition liquids to the patient through the nutrition containers 202, inlet tube 206, and lower esophageal stoma 304. The display 600 can also include an outlet panel that displays information and receives inputs regarding the collection of pre-digested food consumed by the patient through the mouth and exits through the upper esophageal stoma, outlet tube 208, and waste container 212.

In certain implementations, the inlet panel includes a mode selection section where the user can select the mode of operation for the controlled food intake apparatus 200. For example, the user can select an auto, manual, or override mode of operation. In addition, the inlet panel displays a currently selected mode of operation. In certain embodiments, an auto mode of operation indicates that the controlled food intake apparatus 200 is operated via control signals sent by the controller 102 based on nutrition intake orders submitted by a prescribing medical provider. In some implementations, the nutrition intake orders include types, quantities, nutrition content, and times of day that nutrition liquids are to be administered to the patient via the controlled food intake apparatus 200. The inlet display can include a start/stop pushbutton for the user to initiate and/or terminate the administration of nutrition liquids to the patient when the controller 102 is in the auto or override mode of operation.

The manual mode of operation indicates that the controller 102 remains passive and does not issue control signals to control the controlled food intake apparatus 200, and the user, such as the medical provider and/or patient, manually manipulates the controlled food intake apparatus 200 to administer nutrition liquids to the patient. For example, in the manual mode of operation, the user manually opens the proportional flow valve via the manual valve operator on the inlet flow controller 204 to a desired degree of openness to start the flow of nutrition liquids through the inlet tube 206. In some implementations, the controller 102 issues warnings and alarms in the manual mode of operation but does not issue control signals to stop the flow of nutrition liquids.

The override mode of operation indicates that the controller 102 issues control signals to control the controlled food intake apparatus 200, but the user, such as the medical provider, can override the nutrition intake order submitted by the prescribing medical provider and set the flow rate, quantity of nutrition liquids to be administered to the patient, and the like. For example, the inlet panel includes arrow selection buttons for the user to raise or lower the flow rate when the controller 102 is in the override mode of operation. In addition, in the override mode of operation, the controller 102 does not stop administering nutrition liquids to the patient when warnings and/or alarms are output. For example, in the override mode of operation, the medical provider and/or patient pushes a start/stop button to open the inlet flow controllers 204 to achieve the desired flow rate, and then pushes the start/stop button to stop administering nutrition liquids to the patient, which closes the inlet flow controllers 204 to stop flow.

The inlet panel of the display 600 can also include a flow rate display that indicates the measured and ordered flow rates for the nutrition containers 202. The inlet panel can also include a level display that shows how full the nutrition containers 202 are. In some implementations, the level of the nutrition containers 202 can be displayed as a number of centimeters or inches from a highest allowable level or as a percentage of maximum allowable fullness. The inlet panel can also display a quantity of nutrition liquids consumed by the patient and a quantity of remaining liquids to be consumed by the patient. In addition, the inlet panel can display the type of nutrition liquids being administered by the patient, which can be determined by the prescribing medical provider, according to certain embodiments.

The inlet panel also includes a message/warning display that displays events that have occurred related to the controlled food intake apparatus 200. For example, the message/warning display can display information regarding times of operation mode selection, feeding commencement, alarms, and the like. An alarm indicator provides a visual indication on the display 600 of the controller 102 that an alarm has been activated. In some implementations, the alarm indicator includes an acknowledgement pushbutton so that the user can silence the alarm by depressing the acknowledgement pushbutton.

The outlet panel of the display includes indications, alarms, and warnings related to the pre-digested food that travels from the patient's mouth, through the esophagus 216, upper esophageal stoma 302, and outlet tube 208 and collected in the waste container 212. The outlet panel can display the flow rate through the outlet tube 208 that is detected by the outlet flowmeter 210. The outlet panel can also display a maximum allowed quantity of food to be consumed by the patient via the mouth as well as the quantity of food consumed since depressing a start/stop pushbutton or detecting flow through the outlet flowmeter 210. The outlet panel can also display a level of the waste container 212 detected by the level detectors which can be indicated as a number of centimeters or inches from a highest allowable level or as a percentage of maximum allowable fullness.

The outlet panel also includes a message/warning display that displays events that have occurred related to the collection of pre-digested food by controlled food intake apparatus 200. For example, the message/warning display can display information regarding times of meal commencement, maximum food intake alarms, waste container maximum capacity alarms, and the like. An alarm indicator provides a visual indication on the outlet panel that an alarm has been activated. In some implementations, the alarm indicator includes an acknowledgement pushbutton so that the user can silence the alarm by depressing the acknowledgement pushbutton.

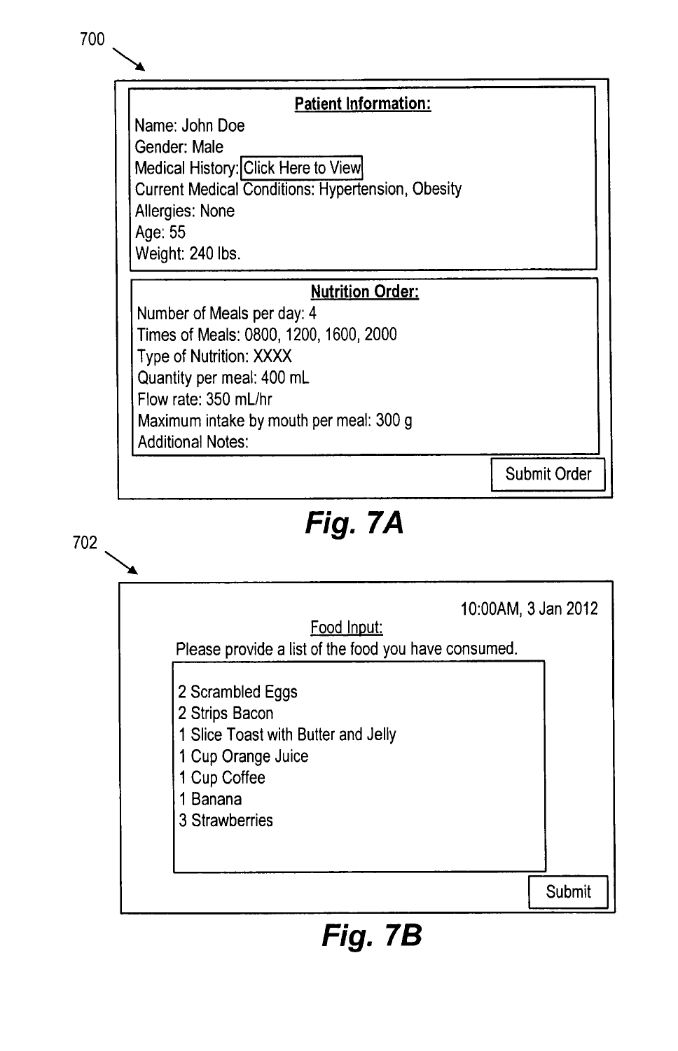

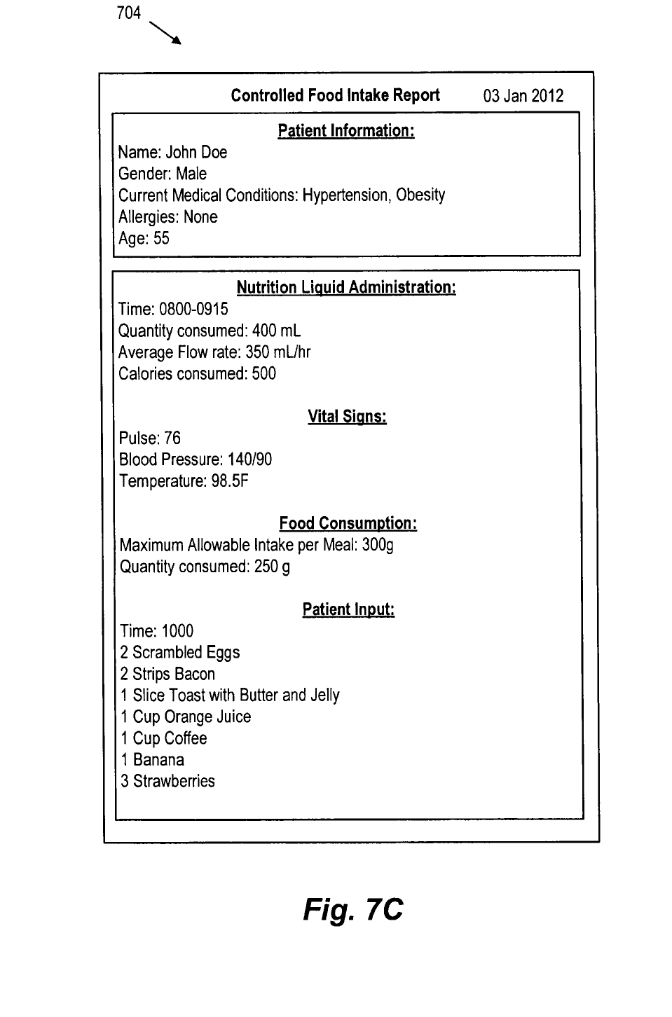

FIG. 7A is an exemplary illustration of a patient order screen 700, according to certain embodiments. The prescribing medical provider, such as a physician or nutritionist, can input orders for nutrition liquids to be administered to the patient via the controlled food intake apparatus 200 via an interface at the computer 110 or via an application at a mobile device 112. In some implementations, the server 106 can retrieve a patient medical record from the database 108 that is displayed to the prescribing medical provider to assist in determining a controlled food intake plan for the patient. For example, the prescribing medical provider can view medical record information that includes the patient's age, gender, medical history, current medical condition, allergies, weight, and the like.

Based on his or her review of the patient medical record, the prescribing medical provider inputs nutrition intake orders at the computer 110 or mobile device 112. The nutrition intake orders can include number of meals per day, times of meals, type of nutrition liquids to be administered, quantity of nutrition liquids to be administered per meal, flow rate, and the like. The nutrition intake orders are transmitted to the server 106 via the network 104 and can be stored in the database 108 for later access by the medical provider and/or patient at the controller 102.

FIG. 7B is an exemplary illustration of a patient meal input screen 702, according to certain embodiments. After the patient consumes food by mouth, he or she can input meal data which includes the types and quantities of food consumed via an interface at the computer 110 or via an application at a mobile device 112. The meal data input by the patient is transmitted to the server 106 via the network 104 and can be stored in the database 108. The meal input data can also be provided in the reports that are generated for the medical providers to receive at the computer 110 and/or mobile device 112.

FIG. 7C is an exemplary illustration of a controlled food intake report 704, according to certain embodiments. The controlled food intake report 704 is generated by the server 106 based on data received from the controller 102 pertaining to the operation of the controlled food intake apparatus 200 as well as information input by the patient and/or medical provider at the computer 110 and/or mobile device 112 as previously discussed. Based on the controlled food intake report 704, the medical provider can assess how well the patient is responding to a dietary plan, how well the patient is at doing at restricting food intake by mouth, and the like. The controlled food intake report can include patient information, nutrition liquid administration information, vital signs, food consumption information, patient inputs, and the like. The patient information includes information that identifies the patient and identifies existing medical conditions. The vital signs can include an average and/or maximum of observed vital signs for the patient during the period of time that the controlled food intake report 704 covers.

In some implementations, the nutrition liquid administration information includes time periods during which nutrition liquids were administered to the patient via the controlled food intake apparatus 200 as well as quantities of nutrition liquids consumed, average flow rates, and calories consumed. The nutrition liquid administration information can also include other nutrition information such as quantities of protein, carbohydrates, and fat consumed by the patient. The food consumption information includes quantities of food consumed through the mouth of the patient that exits the patient's body through the upper esophageal stoma 302 and outlet tube 208 and is collected in the waste container 212. The food consumption information can also include a maximum allowable intake per meal. The controlled food intake report 704 can also include types and quantities of foods input by the patient at the patient meal input screen 702.

The controlled food intake apparatus 200 enables patients who are suffering from medical conditions, such as obesity, hypertension, diabetes, and the like, to receive nutrients to reduce the effects of the medical conditions via the nutrition containers 202 and the inlet tube 206 without having to modify existing dietary habits due to the pre-digested food exiting the body through the upper esophageal stoma 302. In some implementations, patients may undergo stress from the psychological impact of having to immediately change existing dietary habits. The controlled food intake apparatus 200 can alleviate the stress by allowing the patient to consume desired foods while still receiving nutrition liquids through the inlet tube 206. The controller 102 manages the administration of nutrition liquids to the patient based on inputs from the prescribing medical provider. In certain embodiments, the patient can be trained to make healthier food choices over time by monitoring the types and quantities of foods that that patient consumes by mouth that are collected in the waste container 212. The medical provider can make recommendations to the patient regarding healthy dietary habits based on the information received regarding the types of food the patient is consuming by mouth.

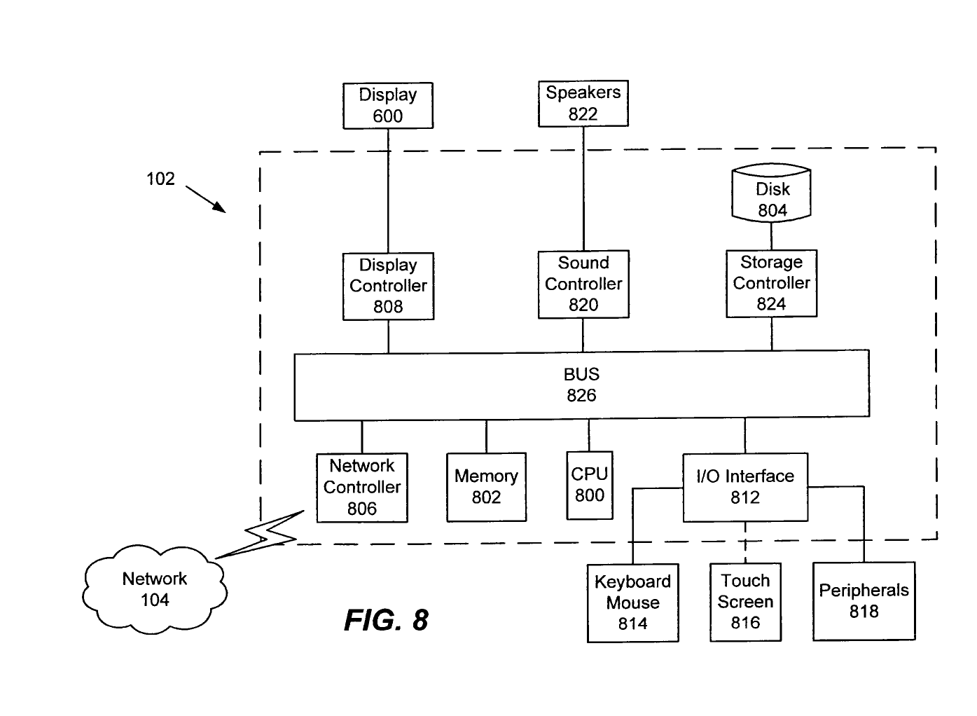

A hardware description of the controlled food intake apparatus controller 102 according to exemplary embodiments is described with reference to FIG. 8. The server 106 also includes one or more of the components described herein as would be understood by one of ordinary skill in the art. The controller 102 includes a CPU 800 that perform the processes described herein. The process data and instructions may be stored in memory 802. These processes and instructions may also be stored on a storage medium disk 804 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the server 106 communicates, such as the computer 110.

Further, the claimed advancements may be provided as a utility application, background daemon, or component of an operating system, or combination thereof, executing in conjunction with CPU 800 and an operating system such as Microsoft Windows 7, UNIX, Solaris, LINUX, Apple MAC-OS and other systems known to those skilled in the art.

CPU 800 may be a Xenon or Core processor from Intel of America or an Opteron processor from AMD of America, or may be other processor types that would be recognized by one of ordinary skill in the art. Alternatively, the CPU 800 may be implemented on an FPGA, ASIC, PLD or using discrete logic circuits, as one of ordinary skill in the art would recognize. Further, CPU 800 may be implemented as multiple processors cooperatively working in parallel to perform the instructions of the inventive processes described above.

The controller 102 in FIG. 8 also includes a network controller 806, such as an Intel Ethernet PRO network interface card from Intel Corporation of America, for interfacing with network 104. As can be appreciated, the network 104 can be a public network, such as the Internet, or a private network such as an LAN or WAN network, or any combination thereof and can also include PSTN or ISDN sub-networks. The network 104 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The controller 102 further includes a display controller 808, such as a NVIDIA GeForce GTX or Quadro graphics adaptor from NVIDIA Corporation of America for interfacing with display 600 of the controller 102, such as a Hewlett Packard HPL2445w LCD monitor. A general purpose I/O interface 812 at the controller 102 or server 106 interfaces with a keyboard and/or mouse 814 as well as a touch screen panel 816 on or separate from display 600. General purpose I/O interface 812 also connects to a variety of peripherals 818 including printers and scanners, such as an OfficeJet or DeskJet from Hewlett Packard.

A sound controller 820 is also provided in the controller 102, such as Sound Blaster X-Fi Titanium from Creative, to interface with speakers/microphone 822 thereby providing sounds and/or music.

The general purpose storage controller 824 connects the storage medium disk 804 with communication bus 826, which may be an ISA, EISA, VESA, PCI, or similar, for interconnecting all of the components of the controller 102. A description of the general features and functionality of the display 600, keyboard and/or mouse 814, as well as the display controller 808, storage controller 824, network controller 806, sound controller 820, and general purpose I/O interface 812 is omitted herein for brevity as these features are known.

In other alternate embodiments, processing features according to the present disclosure may be implemented and commercialized as hardware, a software solution, or a combination thereof. Moreover, instructions corresponding to the controlled food intake process 400 in accordance with the present disclosure could be stored in a thumb drive that hosts a secure process.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes and algorithms described herein may be performed in hardware or software executed by hardware, including computer processors and/or programmable circuits configured to execute program code and/or computer instructions to execute the functions, processes and algorithms described herein. Additionally, an implementation may be performed on modules or hardware not identical to those described. Accordingly, other implementations are within the scope that may be claimed.

The above disclosure also encompasses the embodiments listed below.

(1) A system for controlled food intake, including: a controlled food intake apparatus, having an inlet tube configured to connect to a lower esophageal stoma, a flow controller, at least one container configured to detachably attach to the inlet tube and to administers nutrition liquids to a patient at a flow rate based on a position of the flow controller, an outlet tube configured to connect to an upper esophageal stoma, at least one waste container configured to detachably attach to the outlet tube, and processing circuitry configured to control the flow controller to control the flow rate of the nutrition liquids being administered to the patient based on prescribed nutrition intake order; and at least one server configured to generate reports based on at least one of the nutrition liquids administered to the patient and substance consumed by the patient.

(2) The system of (1), wherein the inlet tube includes an inlet flowmeter downstream of the flow controller to detect a measured flow rate of fluid passing through the inlet tube.

(3) The system of (1) or (2), wherein the processing circuitry is further configured to output an alarm when a difference between the ordered flow rate and the measured flow rate is greater than a predetermined threshold.

(4) The system of any one of (1) to (3), wherein the processing circuitry is further configured to determine a quantity of the nutrition liquids that have been administered to the patient.

(5) The system of any one of (1) to (4), wherein the at least one server is further configured to receive inputs from a medical provider regarding the prescribed nutrition intake orders.

(6) The system of any one of (1) to (5), wherein one or more inflators are detachably attached to proximal ends of the inlet tube and the outlet tube to establish seals with an esophagus of the patient when the inlet tube is inserted into the lower esophageal stoma and the outlet tube is inserted into the upper esophageal stoma.

(7) The system of any one of (1) to (6), wherein the processing circuitry is further configured to detect when a pressure in the inflators is less than a predetermined pressure.

(8) The system of any one of (1) to (7), wherein the processing circuitry is further configured to determine a minimum starting level for the at least one container to provide a prescribed quantity of the nutrition liquids to the patient.

(9) The system of any one of (1) to (8), wherein the processing circuitry is further configured to output an alarm when an initial level of the at least one container is less than the minimum starting level.

(10) The system of any one of (1) to (9), wherein the processing circuitry is further configured to output an alarm when the at least one container is at a predetermined empty value.

(11) The system of any one of (1) to (10), wherein the processing circuitry is further configured to determine when a prescribed quantity of nutrition liquids has been administered to the patient.

(12) The system of any one of (1) to (11), wherein the processing circuitry is further configured to override the prescribed nutrition intake orders in an override mode of operation based on predetermined inputs.

(13) The system of any one of (1) to (12), wherein the processing circuitry is further configured to ignore one or more alarms when in the override mode of operation.

(14) The system of any one of (1) to (13), wherein the nutrition intake orders include a quantity of the nutrition liquids to be administered to the patient, the ordered flow rate of the nutrition liquids through the inlet tube, and a maximum allowable intake of substance.

(15) The system of any one of (1) to (13), wherein the outlet tube includes an outlet flowmeter that detects an outlet tube flow rate of substance passing through the outlet tube.

(16) The system of any one of (1) to (15), wherein the processing circuitry is further configured to output an alarm when the outlet flowmeter detects flow through the outlet tube at an unauthorized time.

(17) The system of any one of (1) to (16), wherein the processing circuitry is further configured to determine a quantity of substance consumed based on at least one of a weight of the waste container and the outlet tube flow rate.

(18) The system of any one of (1) to (17), wherein the processing circuitry is further configured to output an alarm when the quantity of substance consumed by the patient is greater than a predetermined maximum allowable intake.

(19) The system of any one of (1) to (18), wherein the processing circuitry is further configured to output an alarm when a quantity of substance in the waste container is greater than a predetermined threshold.

(20) An apparatus for controlled food intake, including: an inlet tube configured to connect to a lower esophageal stoma; a flow controller; at least one container configured to detachably attach to the inlet tube and to administers nutrition liquids to a patient at a flow rate based on a position of the flow controller; an outlet tube configured to connect to an upper esophageal stoma; at least one waste container configured to detachably attach to the outlet tube and to receive substance; and processing circuitry configured to control the flow controller to control the flow rate of the nutrition liquids being administered to the patient based on prescribed nutrition intake order.

1. A system for controlled food intake, comprising:
a controlled food intake apparatus, having
an inlet tube configured to connect to a lower esophageal stoma,
a flow controller,
at least one container configured to detachably attach to the inlet tube and to administer nutrition liquids to a patient at a flow rate based on a position of the flow controller,
an outlet tube configured to connect to an upper esophageal stoma,
at least one waste container configured to detachably attach to the outlet tube, and
processing circuitry configured to control the flow controller to control the flow rate of the nutrition liquids being administered to the patient based on prescribed nutrition intake order; and
at least one server configured to generate reports based on at least one of the nutrition liquids administered to the patient and substance consumed by the patient.

2. The system of claim 1, wherein the inlet tube includes an inlet flowmeter downstream of the flow controller to detect a measured flow rate of fluid passing through the inlet tube.

3. The system of claim 2, wherein the processing circuitry is further configured to output an alarm when a difference between the ordered flow rate and the measured flow rate is greater than a predetermined threshold.

4. The system of claim 1, wherein the processing circuitry is further configured to determine a quantity of the nutrition liquids that have been administered to the patient.

5. The system of claim 1, wherein the at least one server is further configured to receive inputs from a medical provider regarding the prescribed nutrition intake orders.

6. The system of claim 1, wherein one or more inflators are detachably attached to proximal ends of the inlet tube and the outlet tube to establish seals with an esophagus of the patient when the inlet tube is inserted into the lower esophageal stoma and the outlet tube is inserted into the upper esophageal stoma.

7. The system of claim 6, wherein the processing circuitry is further configured to detect when a pressure in the inflators is less than a predetermined pressure.

8. The system of claim 1, wherein the processing circuitry is further configured to determine a minimum starting level for the at least one container to provide a prescribed quantity of the nutrition liquids to the patient.

9. The system of claim 8, wherein the processing circuitry is further configured to output an alarm when an initial level of the at least one container is less than the minimum starting level.

10. The system of claim 1, wherein the processing circuitry is further configured to output an alarm when the at least one container is at a predetermined empty value.

11. The system of claim 1, wherein the processing circuitry is further configured to determine when a prescribed quantity of nutrition liquids has been administered to the patient.

12. The system of claim 1, wherein the processing circuitry is further configured to override the prescribed nutrition intake orders in an override mode of operation based on predetermined inputs.

13. The system of claim 12, wherein the processing circuitry is further configured to ignore one or more alarms when in the override mode of operation.

14. The system of claim 1, wherein the nutrition intake orders include a quantity of the nutrition liquids to be administered to the patient, the ordered flow rate of the nutrition liquids through the inlet tube, and a maximum allowable intake of substance.

15. The system of claim 1, wherein the outlet tube includes an outlet flowmeter that detects an outlet tube flow rate of substance passing through the outlet tube.

16. The system of claim 15, wherein the processing circuitry is further configured to output an alarm when the outlet flowmeter detects flow through the outlet tube at an unauthorized time.

17. The system of claim 15, wherein the processing circuitry is further configured to determine a quantity of substance consumed based on at least one of a weight of the waste container and the outlet tube flow rate.

18. The system of claim 17, wherein the processing circuitry is further configured to output an alarm when the quantity of substance consumed by the patient is greater than a predetermined maximum allowable intake.

19. The system of claim 1, wherein the processing circuitry is further configured to output an alarm when a quantity of substance in the waste container is greater than a predetermined threshold.

20. An apparatus for controlled food intake, comprising:
an inlet tube configured to connect to a lower esophageal stoma;
a flow controller;
at least one container configured to detachably attach to the inlet tube and to administers nutrition liquids to a patient at a flow rate based on a position of the flow controller;
an outlet tube configured to connect to an upper esophageal stoma;
at least one waste container configured to detachably attach to the outlet tube and to receive substance; and
processing circuitry configured to control the flow controller to control the flow rate of the nutrition liquids being administered to the patient based on prescribed nutrition intake order.
